(19) United States
(12) Patent Application Publication
(10) Pub. No.: US 2010/0119077 A1
(43) Pub. Date: May 13, 2010

(54) ACTIVE HEARING PROTECTION SYSTEM

(75) Inventors: Rainer Platz, Colombier (CH); Olivier Hautier, Savagnier (CH)

Correspondence Address: ROBERTS MLOTKOWSKI SAFRAN & COLE, P.C.;Intellectual Property Department, P.O. Box 10064, MCLEAN, VA, 22102-8064, US

(73) Assignee: PHONAK AG, Staefa (CH)

(21) Appl. No.: 12/519,961

(22) PCT Filed: Dec. 18, 2006
(86) PCT No.: PCT/EP06/12201

§ 371 Date: Jan. 18, 2010

(51) Int. Cl. A61F 11/06

(52) U.S. Cl. 381/72

(57) ABSTRACT

An active hearing protecting system right earplug and a left earplug and a wearable central unit to be worn at the user's body a digital audio signal processing unit which has at least two channels and which can be connected via an audio input to an external audio signal source each earplug including a microphone and a loudspeaker which are connected to the audio signal processing unit. The central unit also has a detector for detecting audio signals and being adapted to control the audio signal processing unit when audio signal is detected. The audio signal processing unit has an ambient mode in which the audio signals captured by the left ear microphone are provided to the left ear loudspeaker and the audio signals captured by the right ear microphone are provided to the right ear loudspeaker, and a communication mode in which the audio signals from the audio input are provided at least one of the loudspeakers and audio signals captured by at least one of the microphones are provided to the other ear loudspeaker.

### BACKGROUND OF THE INVENTION

1. Field of the Invention

The present invention relates to an active haring protection system comprising a left and a right earplug, each being adapted for providing for an acoustic attenuation of at least 10 dB averaged over the audible frequency range and comprising an active unit with a microphone for capturing audio signals from ambient sound and a loudspeaker.

2. Description of Related Art

U.S. Pat. No. 4,677,678 relates to a hearing protection system comprising two earmuffs or earplugs comprising each a microphone and a speaker. The system comprises two audio channels sharing a common sound level limiting unit.

US 2002/0080979 A1 relates to a hearing protection system comprising two earplugs into which an active unit comprising each a microphone and a speaker is inserted. The gain as a function of the sound input level is a three-segment piece-wise linear function including a first section providing maximum expansion up to a first knee point for maximum noise reduction, a second section providing less expansion up to a second knee point for less noise reduction, and a third section providing minimum or no expansion for input signals with high signal-to-noise-ratio (SNR) to minimise distortion.

EP 1 674 061 A1 relates to a hearing protection system comprising two earplugs including an active unit comprising a microphone and a speaker, wherein the audio signal processing mode is controlled by sound classification including level analysis. If the acoustic noise level is high, the gain may be significantly reduced or even completely eliminated. The system includes a transparent gain setting if acoustic noise is low, and a close speech mode if close speech in acoustic noise has been detected.

EP 1 674 057 A1, EP 1 615 468 A1 and DE 101 17 705 A1 relate to hearing protection systems comprising a microphone, an audio signal processing unit and a speaker at each ear, wherein in addition a wireless interface for an external audio source, such as a mobile phone or a remote active hearing protection system used by another person, is provided.

Hearing protection systems provided with an interface for wireless audio input from a remote audio signal source, but not having microphones at ear level, are known, for example, from EP 1 674 059 A1, GB 2 373 951 A, U.S. Pat. No. 5,426,719 and US 2003/0059071 A1.

EP 1 653 773 A2 relates to a hearing aid which is capable of automatically selecting, for example according to the SNR or according to the audio signal level, the audio signal input from one of a plurality of audio signal sources. The audio signal sources may be a T-coil receiver, a frequency modulation (FM) radio receiver or a direct audio input. The hearing aid also comprises a classifier for selecting the audio signal processing mode of the hearing aid according to the analysis of the auditory scene. A similar hearing aid is described in EP 1 443 803 A2.

U.S. Pat. No. 5,721,783 relates to a hearing instrument comprising two ear-pieces with a microphone and a speaker and a remote audio signal processing unit wirelessly connected to the ear-pieces. Audio signals from a phone can be provided via the remote audio signal processing unit to the ear-pieces. A switch is provided for switching between the microphone signals and the phone signals as the audio input to audio signal processing unit. The system may comprise active noise cancelling capability.

US2004/136522 A1 relates to an in-the-ear headset comprising an active noise reduction arrangement and a communication loudspeaker and a boom microphone. The communication loudspeaker and a boom microphone may be connected, via a controller, to a primary and a secondary communication unit for exchanging audio signals with another person. The controller detects the presence of the primary and the secondary communication unit and controls the weight of the audio signals from the primary and the secondary communication unit.

It is an object of the invention to provide for an active hearing protection system which provides for a particularly comfortable and flexible communication function. It is a further object to provide for a corresponding method for providing hearing protection to a user.

### SUMMARY OF THE INVENTION

According to the invention, these objects are achieved by an active hearing protection system as defined in claim 1 and a method as defined in claim 39, respectively. The invention is beneficial in that, by providing for a central unit having a digital audio signal processing unit having at least two channels and by automatically controlling the audio signal processing unit depending on whether the presence of an audio signal at the audio input of the audio signal processing unit has been detected or not, external audio signals can be combined for communication purposes in a particularly comfortable and flexible manner with the audio signal captured by the microphones of the system.

Preferred embodiments of the invention are defined in the dependent claims.

These and further objects, features and advantages of the present invention will become apparent from the following description when taken in connection with the accompanying drawings which, for purposes of illustration only, show several embodiments in accordance with the present invention.

### BRIEF DESCRIPTION OF THE DRAWINGS

FIG. 1 is a schematic view of an example of an active hearing protection system according to the invention;

FIG. 2 is a block diagram of the electronic components of the system of FIG. 1;

FIG. 3 is a block diagram of the system wherein operation in the communication mode is illustrated; and

FIG. 4 is a diagram showing an example of the acoustic gain model of the system.

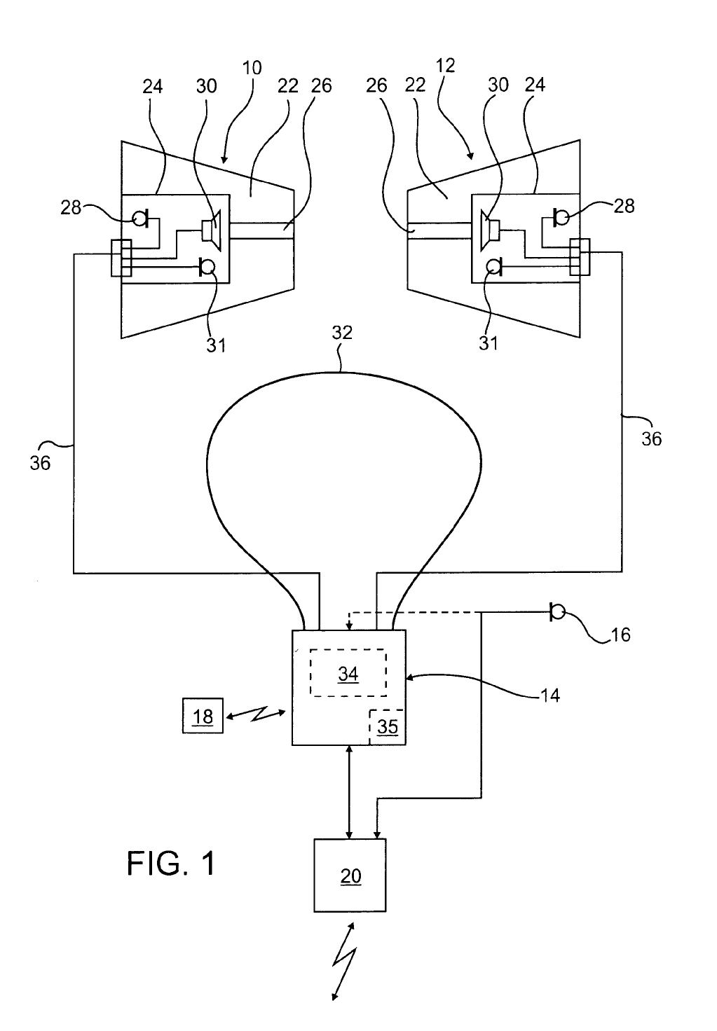

The active hearing protection system shown in FIG. 1 comprises a left ear earplug 10, a right ear earplug 12, a central unit 14, a boom microphone 16 and a remote control unit 18. The central unit 14 is connected to a communication device 20.

### DETAILED DESCRIPTION OF THE INVENTION

Preferably the hearing protection earplugs 10, 12 have a hard shell with an elasticity from shore D85 to shore D65, which is customized, i.e. it has an outer shape according to the individual measured inner shape of the user's outer ear and ear canal. The hard shell 22 may be manufactured by layer-by-layer laser sintering of a powder material, for example, polyamide powder or by laser stereo-lithography or photo-polymerization. An overview regarding such additive layer-by-layer build-up processes for manufacturing customized shells of hearing devices can be found, for example, in US 2003/0133583 A1 or U.S. Pat. No. 6,533,062 B1. The inner shape of the user's ear canal and outer ear can be measured, for example, by taking an impression which then undergoes laser scanning or by direct laser scanning of the ear. Preferably the hard shell 22 is designed such that it provides for an acoustic attenuation of at least 10 dB averaged over the audible frequency range when worn by the user.

The shell 22 has a receptacle into which an active unit 24 can be inserted, preferably in a releasable manner. The shell 22 is provided with a sound channel 26 through which the active unit 24 is acoustically connected to the ear canal. The active unit 24 comprises a microphone 28 for capturing audio signals from ambient sound and a loudspeaker 30 for providing audio signals into the user's ear canal via the sound channel 26. Such earplugs comprising an active unit are described, for example, in EP 1 674 059 A1.

The central unit 14 is to be worn at the user's body, e.g. by a loop 32 around the user's neck. The central unit 14 comprises an audio signal processing unit 34 for receiving and processing the audio signals captured by the microphones 28 and for supplying the loudspeakers 30 with audio signals to be reproduced to the user's ear. To this end, the active units 24 are connected to the central unit 14 via cable connections 36. The remote control unit 18 is connected to the central unit 14 via a wireless link in order to serve as a user interface.

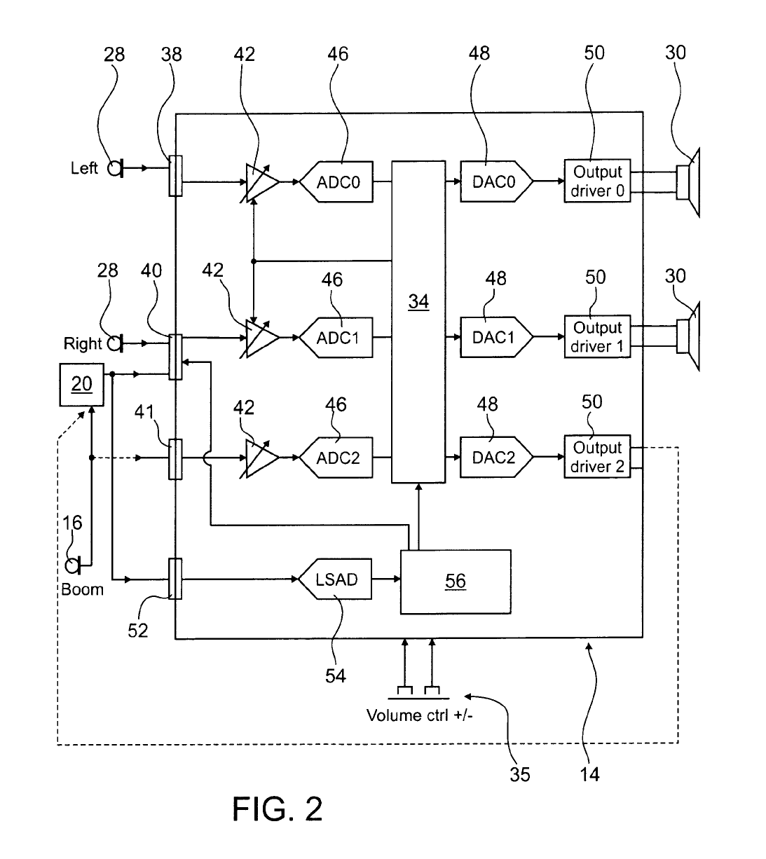

The boom microphone 16 may be connected to the central unit in order to supply audio signals captured from the user's voice (dashed lines in FIGS. 1 and 2). Alternatively, it may be connected directly to the communication device 20. The communication device 20, for example, may be a mobile phone or a FM radio device, such as a walkie-talkie. Usually the communication device 20 and the central unit 14 will be connected by wires, although connection by a wireless link is conceivable.

In order to act as an active hearing protection system, audio signals captured from ambient sound by the microphones 28 are provided, after processing in the audio signal processing unit 34, to the speakers 30 in order to provide sound impinging on the left ear to the left ear canal and sound impinging on the right ear to the right ear canal despite the acoustic attenuation provided by the shell 22 for relatively low sound pressure levels (for high sound pressure levels the gain will be progressively reduced in order to provide for a hearing protection function). Further, audio signals received from the communication device 20 are also to be provided to the user's ear via the central unit 14 and the speakers 30. In addition, if bidirectional communication is desired, the user's voice can be captured by the boom microphone 16 and/or the microphones 28, 31 and will be supplied to the communication device 20.

The structure and the functionality of the central unit 14 will be explained in more detail by reference to FIGS. 2 to 4. The central unit 14 comprises an input 38 for the left ear microphone 28, an input 40 for the right ear microphone 28 and the audio signals from the communication device 20 and optionally an input 41 for the boom microphone 16. Each of these channels is provided with a pre-amplifier 42 which preferably is adjustable. The pre-amplified signal is digitized in an analogue-to-digital converter 46 before it is supplied to the audio signal processing unit 34, which has at least two outputs for outputting corresponding the processed digital audio signals to a digital-to-analogue converter 48, with the respective analogue signal being provided to an output driver 50 driving the left ear speaker 30 and the right ear speaker 30, respectively. A third output comprising a digital-to-analogue converter 48 and an output driver 50 may be provided for supplying audio signals from the audio signal processing unit 34 to the communication device 20.

The central unit 14 comprises a further audio input 52 which is connected in the same manner as the input 40 to the communication device 20 and hence receives the same audio signals. The audio signals received at the input 52 are provided to a low speed analogue-to-digital converter 54, with the digitized signals being provided to a detector 56 for detecting the presence of an audio signal from the communication device 20 at the audio input 52. On the one hand, the detector 56 supplies a status signal to the audio signal processing unit 34 in order to select the audio signal processing mode depending on whether there is an audio signal from the communication device 20 or not. On the other hand, the detector 56 also acts on the audio input 40 which can be switched between the microphone 28 and the communication device 20 depending on whether an audio signal from the communication device 20 is present or not.

If no audio signal from the communication device 20 is detected by the detector 56, the audio signal processing unit 34 operates in an “ambient mode” in which the right ear microphone 28 is selected as the audio signal source to the input 40 rather than the communication device 20. In the ambient mode the audio signal processing unit 34 primarily acts as a stereo device, i.e. the audio signals from the right ear microphone 28 are supplied essentially to the right ear speaker 30, and the audio signals from the left ear microphone 28 are essentially supplied to the left ear speaker 30. However, there may be some transfer of audio signals between the two channels in order to enable binaural audio signal processing. Examples of binaural audio signal processing can be found in EP 1 320 281 A2, WO 99/43185 or US 2004/02 52852 A1. Apart from such binaural processing, both channels usually will be processed in the same manner, if the user is a normal hearing person. If, however, the user suffers from asymmetric hearing loss, also the audio signal processing in the “ambient mode” will be asymmetric in order to compensate for the hearing loss as far as possible.

In FIG. 4 an example of the acoustic gain model implemented in the audio signal processing unit 34 is shown, wherein the output free field sound pressure level (SPL FF) is given as a function of the input SPL FF. In the example shown in FIG. 4 it is assumed that the shell 22 provides for an attenuation of 30 dB. The curve with the diamonds represents the situation in which the gain is set to zero (i.e. no sound from the speakers 30). On this curve, the hearing protection system acts as a passive system.

The curve with the squares represents the situation in which the system has a “transparent” gain, i.e. below the knee point (which is set to 70 dBA) the user has the same sound feeling as if he was not wearing the earplugs 10, 12. Different gain settings are applied below and above the knee point (which is set to 70 dBA in the example of FIG. 4). Below this knee point the gain is constant, resulting in a linear curve. Above the knee point, i.e. for higher input levels, the gain is progressively reduced so that the output level remains constant. For even higher input levels the system will act as a passive hearing protector.

The system also comprises a manual volume control which is implemented on the central unit 14 or, alternatively or additionally, in the remote control unit 18. By actuating the volume control, the value of the constant gain applied in the linear region below the knee point can be adjusted between a minimum value (circles in FIG. 4) and a maximum value (triangles in FIG. 4). When the maximum gain is applied, the maximum output free field sound pressure level is limited to 80 dBA SPL FF in the example of FIG. 4. 85 dBA SPL FF correspond to the maximum noise level tolerable for 8 hours a day according to Directive 2003/10/EC. Since the open ear gain (OEG) is around 10 dB, the sound pressure level in the ear canal is around 10 dB higher than the free-field values given in FIG. 4.

In FIG. 4 the gain model is shown for one frequency. The gain model to be applied by the audio signal processing unit 34 may be frequency-dependant in order to compensate for the frequency-dependency of the acoustic attenuation provided by the shell 22 and/or a hearing loss of the user.

Beyond the volume control interface, the central unit 14 may comprise manual user interfaces such as e.g. on/off, audio signal processing mode (program) change, push-to-talk (PTT), call accept/reject or voice dialing. These manual control interfaces are designated by 35 in FIGS. 1 and 2. The same commands or a subset thereof may be provided on the remote control unit 18.

The acoustic gain model may be automatically selected according to the result of an auditory scene analysis based on the audio signals provided by the microphones 28.

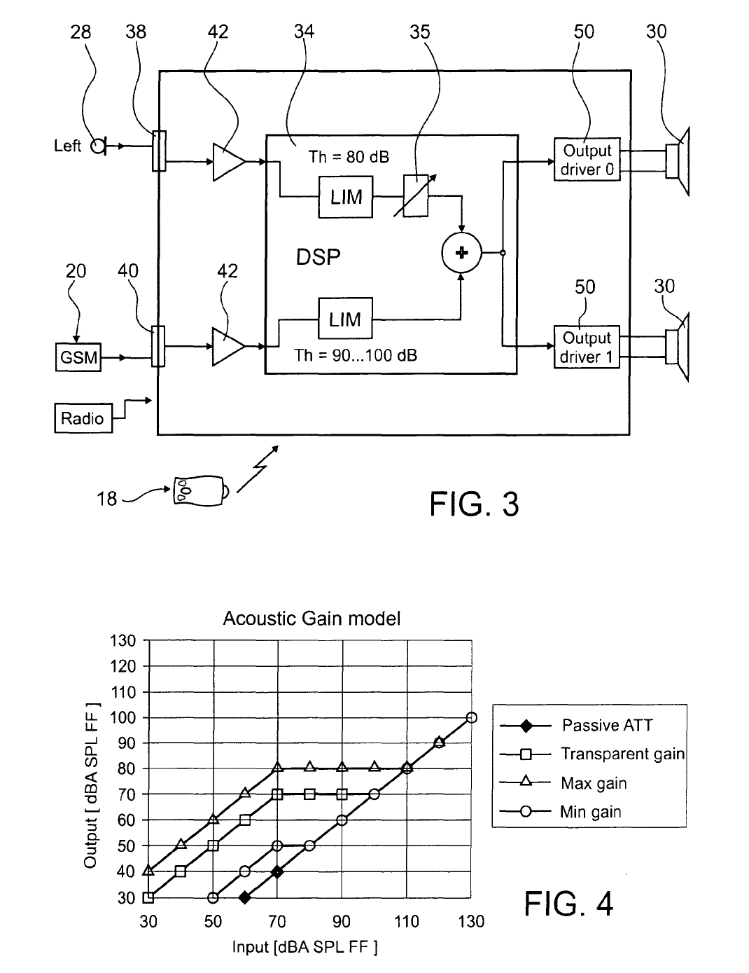

Once an audio signal from the communication device 20 is detected by the detector 56, the central unit 14 will switch into a “communication mode” in which the input from the right ear microphone 28 is replaced by the audio signals provided by the communication device 20. Whereas in the ambient mode—apart from compensation of an asymmetric hearing loss—usually the same gain model is applied to both channels, in the communication mode preferably a different gain model now is applied to that channel to which the audio signals from the communication device 20 are provided to. In this “communication channel” the maximum output free field sound pressure level may be shifted to higher values, for example in such a manner that an output level of 100 dBA SPL FF is allowed (rather than 80 dBA SPL FF allowable for the channel of the left ear). This level limiting function in both channels is labelled “LIM” (“limiter”) in FIG. 3. In the communication mode the volume control will act only on the “ambient channel”, i.e. the left ear microphone channel. After having undergone respective audio signal processing, the two channels may be combined and then may be supplied as a mono signal to both loudspeakers 30.

Such an example of operation of the system in the communication mode is schematically shown in FIG. 3 (the right ear microphone 28 is omitted in FIG. 3, since it is blocked by action of the detector 56 on the input 40). According to this example in the communication mode a mixture of audio signals representative of the ambient sound as captured by the left ear microphone 28 and the audio signals provided by the communication device 20 is provided to both ears. The user can adjust the relative level of the “ambient channel” and the “communication channel” by actuating the volume control on the central unit 14 or via the remote control unit 18, which in the communication mode acts only on the “ambient channel”.

The volume of the communication channel may be adjusted on the communication device 20.

In case that the audio signals provided by the communication device 20 are speech signals the “communication channel” may undergo some speech enhancement filtering in order to improve intelligibility of the speech. Enhancement of the intelligibility of the voice in the “communication channel” can be achieved, for example, by reducing the audio band width and/or emphasizing speech frequencies (300 Hz to 3.4 kHz). However, such filtering also my be applied to the “ambient channel” or to both channels in the ambient mode if speech signals are received via the microphones 28. The decision that speech is present may be made by performing auditory scene analysis in the audio signal processing unit 34 based on the audio signals captured by the microphones 28.

As alternatives to the above-described example, in the communication mode the audio signals received from the communication device 20 may be provided completely or at least primarily to one of the left ear and right ear loudspeakers 30 and the audio signals captured by at least one the left ear and right ear microphones 28 may be provided completely or at least primarily to the other one of the loudspeakers 30.

According to another alternative, in the communication mode the audio signals from the communication device 20 may be mixed to at least one of the audio signals captured by the left ear microphone 28 and the right ear microphone 28, with, as in the ambient mode, the audio signals captured by the left ear microphone 28 being provided to the left ear speaker and the audio signals from the right ear microphone 28 being provided to the right ear speaker 30. The mixing ratio may be different for the two channels. In this case the central unit would be equipped with a further fast analogue-to-digital converter, so that the audio signals from the communication device 20 could be processed in parallel to the audio signals from the left ear microphone 28 and the right ear microphone 28.

Preferably the interface between the communication device 20 and the central unit 14 is bidirectional so that not only audio signals can be supplied from the communication device 20 to the central unit 14 but also audio signals and/or command signals can be sent from the central unit 14 to the communication device 20. For example, the user's voice may be captured by the boom microphone 16 and/or the microphones 28 in order to be supplied to the communication device 20 via the third output (dashed lines).

The boom microphone 16 may be omitted if the central unit 14 is capable of performing blind source separation (BSS) on the audio signals provided by the microphones 28, 31 in order to separate the user's voice from background noise. Typically, the inner microphone 31 is required on only one of both sides. Preferably, each active unit 24 of the earplugs 10, 12 in this case may include a second microphone 31 which is acoustically oriented towards the ear canal in order to capture primarily the user's voice, whereas the microphone 28 is acoustically oriented towards ambience in order to capture primarily ambient sound.

Alternatively, one or two microphones 31 on the right and/or the left side may pick up the user's voice transferred by bone conduction to the ear canal (in-ear voice pickup).

Alternatively or in addition, the central unit 14 may be capable of performing acoustic beamforming on the audio signals captured by the microphones 28 in order to separate the user's voice from ambient sound/background noise.

The central unit 14 and/or remote control unit 18 preferably comprises a PTT (Push to Talk) element operable by the user to control transmission of audio signals, representing the user's voice, via the communication device 20.

The central unit 14 and/or remote control unit 18 also may comprise a call accept/call reject element and a voice dialing activation element for the event that the communication device 20 is a mobile phone.

The communication device 20 also may serve to supply power to the central unit 14. Alternatively, the central unit 14 may be provided with a primary or rechargeable battery (not shown in FIGS. 1 to 3).

The audio signals representing the user's voice captured by the microphones 28 and/or the boom microphone 16 may be supplied not only to the communication device 20 but also to some extent to the right ear and left ear speaker 30 in order to reduce the occlusion effect caused by the acoustic attenuation of the shell 22.

The central unit 14 may be capable of user preference learning in order to learn and store the preferred manual settings by the user.

Preferably, the audio signal processing unit 34 is realized as a single digital signal processing (DSP) chip.

The central unit 14 also may comprise a noise dosimeter functionality, with the sound exposure of the user being estimated from the level of the audio signals captured by the left ear and right ear microphones 28 or 31, which is integrated over a given time period. The central unit 14 may comprise an alarm function wherein the user is alerted via at least one of the speakers 30 when a given maximum noise dose has been reached. Alternatively or in addition, the central unit 14 may comprise a protection function wherein the gain applied in the audio signal processing unit 34 is reduced when a given maximum noise dose has been reached.

While various embodiments in accordance with the present invention have been shown and described, it is understood that the invention is not limited thereto, and is susceptible to numerous changes and modifications as known to those skilled in the art. Therefore, this invention is not limited to the details shown and described herein, and includes all such changes and modifications as encompassed by the scope of the appended claims.

1-39. (canceled)

40. An active hearing protecting system comprising a right earplug to be worn at least in part in the right ear canal of a user and a left earplug to be worn at least in part in the left ear canal of the user and a central unit to be worn at the user's body and comprising a digital audio signal processing unit which has at least two channels and which can be connected via an audio input to an external audio signal source,
wherein each earplug is adapted for providing for an acoustic attenuation of at least 10 dB averaged over the audible frequency range and comprises an active unit comprising a microphone for capturing audio signals from ambient sound and a loudspeaker which are connected to the audio signal processing unit,
wherein the central unit comprises a detector for detecting the presence of an audio signal at the audio input,
and wherein the central unit is adapted to control the audio signal processing unit in such a manner that, during times when no presence of an audio signal at the audio input is detected, the audio signal processing unit is operated in an ambient mode in which the audio signals captured by the left ear microphone are provided to the left ear loudspeaker and the audio signals captured by the right ear microphone are provided to the right ear loudspeaker, and during times when the presence of an audio signal at the audio input is detected, the audio signal processing unit is operated in a communication mode in which the audio signals received via the audio input are provided to at least one of the left ear loudspeaker and the right ear loudspeaker and the audio signals captured by at least one of the left ear microphone and the right ear microphone are provided to at least the other one of the left ear loudspeaker and the right ear loudspeaker.

41. The system of claim 40, wherein in the communication mode the audio signals received via the audio input and the audio signals captured by one of the left ear microphone and the right ear microphone are mixed and then supplied to both the left ear loudspeaker and the right ear loudspeaker.

42. The system of claim 40, wherein in the communication mode the audio signals received via the audio input are provided completely or at least primarily to one of the left ear loudspeaker and the right ear loudspeaker and the audio signals captured by at least one of the left ear microphone and the right ear microphone are provided completely or at least primarily to the other one of the left ear loudspeaker and the right ear loudspeaker.

43. The system of claim 40, wherein in the communication mode the audio signals captured by the left ear microphone are provided to the left ear loudspeaker and the audio signals captured by the right ear microphone are provided to the right ear loudspeaker, and wherein the audio signals received via the audio input are mixed to at least one of the audio signals captured by the left ear microphone and the audio signals captured by the right ear microphone.

44. The system of claim 43, wherein the mixing ratio is different for mixing with the audio signals captured by the left ear microphone and for mixing with the audio signals captured by the right ear microphone.

45. The system of claim 40, wherein at least one of the parameters used for audio signal processing in the audio signal processing unit is different for the ambient mode and the communication mode for at least one of the channels.

46. The system of claim 45, wherein the parameter is a gain as a function of the input level.

47. The system of claim 40, wherein both in the ambient mode and in the communication mode for the two channels of the audio signal processing unit the gain is progressively reduced above a given threshold value of the input level.

48. The system of claim 47, wherein the gain is essentially independent of the input level for input levels below the threshold value.

49. The system of claim 47, wherein for input levels below the threshold value the gain of at least one of the channels is adjustable by a manual volume control.

50. The system of claim 47, wherein in the ambient mode the gain of both channels is adjustable in parallel by the manual volume control.

51. The system of claim 50, wherein in the communication mode only the gain of that channel carrying primarily the audio signals captured by at least one of the left ear microphone and right ear microphone is adjustable by the manual volume control.

52. The system of claim 47, wherein in the communication mode the maximum acoustic output level is different for the two channels.

53. The system of claim 40, wherein in the ambient mode the gain function of the audio signal processing unit is manually selectable or is automatically selected based on the result of an auditory scene analysis.

54. The system of claim 40, wherein the central unit comprises a bidirectional interface to a wireless communication device, which communication device is for providing audio signals to the audio input, wherein the system comprises means for capturing audio signals to be supplied to the communication device and wherein the system comprises a remote control unit adapted for manual control of the communication device.

55. The system of claim 40, wherein the central unit comprises a bidirectional interface to a wireless communication device, which communication device is for providing audio signals to the audio input, wherein the system comprises means for capturing audio signals to be supplied to the communication device and wherein the central unit comprises a user interface for manual control of the communication device.

56. The system of claim 54, wherein the bidirectional interface is wireless.

57. The system of claim 54, wherein the means for capturing audio signals is a boom microphone which is to be worn by the user to capture the user's voice and which is electrically connected to the communication device directly or via said interface.

58. The system of claim 54, wherein the means for capturing audio signals is at least one of the left ear microphone, the right ear microphone and at least one inner microphone acoustically oriented inwardly towards the ear canal.

59. The system of claim 58, wherein a blind-source-separation unit is included in the audio signal processing unit in order to have the audio signals captured by at least one of the left ear microphone, the right ear microphone and at least one inner microphone undergo a blind-source-separation algorithm in order to separate the user's voice from ambient sound.

60. The system of claim 58, wherein the audio signal processing unit is capable of performing acoustic beamforming on the audio signals captured by the left ear microphone and the right ear microphone in order to separate the user's voice from ambient sound.

61. The system of claim 54, wherein the remote control unit is a handheld unit connected to the central unit by a wireless link.

62. The system of claim 54, wherein at least one of the central unit and the remote control unit comprises a Push-to-talk element for controlling transmission of the audio signals supplied by the audio signal capturing means by the communication device.

63. The system of claim 54, wherein the remote control unit comprises a volume control element for controlling the gain applied by the audio signal processing unit.

64. The system of claim 54, wherein the communication device is a mobile phone, and wherein the remote control unit comprises a call accept/call reject element and a voice dialing activation element.

65. The system of claim 55, wherein the communication device is a mobile phone, and wherein the central unit comprises a call accept/call reject element and a voice dialing activation element.

66. The system of claim 54, wherein power is supplied from the communication device to the central unit via the bidirectional interface.

67. The system of claim 40, wherein the audio signal processing unit in the ambient mode is adapted to enhance by filtering the user's voice in the audio signals captured by the left ear microphone and the right ear microphone or in the audio signals captured by a boom microphone, with the filtered audio signals being provided to the right ear and left ear loudspeaker in order to reduce occlusion effect.

68. The system of claim 40, wherein the central unit is capable of user preference learning in order to learn and store the preferred manual settings by the user.

69. The system of claim 40, wherein the audio signal processing unit is a single DSP chip.

70. The system of claim 40, wherein the central unit comprises a noise dosimeter functionality, with the sound exposure of the user being estimated from the level of the audio signals captured by at least one of the left ear, the right ear microphone and at least one inner microphone acoustically oriented inwardly towards the ear canal and integrated over a given time period.

71. The system of claim 70, wherein the central unit comprises an alarm function wherein the user is alerted via at least one of the left ear and right ear loudspeaker when a given maximum noise dose has been reached.

72. The system of claim 70, wherein the central unit comprises a user protection function wherein the gain applied in the audio signal processing unit is reduced when a given maximum noise dose has been reached.

73. The system of claim 40, wherein the gain applied by the audio signal processing unit in at least one of the ambient mode and the communication mode is frequency-dependent in order to compensate for at least one of the frequency dependence of the acoustic attenuation provided by the earplugs and a hearing loss of the user.

74. The system of claim 40, wherein the gain applied by the audio signal processing unit in at least one of the ambient mode and the communication mode is frequency-dependent in order to enhance speech intelligibility of the audio signals received via the audio input.

75. The system of claim 40, wherein in the ambient mode one of a plurality of audio signal processing modes of the audio signal processing unit is automatically selected according to the result of an auditory scene analysis.

76. The system of claim 40, wherein in the ambient mode one of a plurality of audio signal processing modes of the audio signal processing unit is manually selectable on the central unit or via a remote control unit.

77. The system of claim 40, wherein each earplug comprises a customized shell into which the active unit is detachably inserted.

78. A method for providing hearing protecting to user, comprising:
wearing a right earplug comprising a microphone and a loudspeaker at least in part in the right ear canal and a left earplug comprising a microphone and a loudspeaker at least in part in the left ear canal in such a manner that each earplug provides for an acoustic attenuation of at least 10 dB averaged over the audible frequency range;
wearing a central unit comprising a digital audio signal processing unit which has at least two channels at the user's body;
connecting an external audio signal source via an audio input to the central unit;
detecting whether an audio signal is present at the audio input or not; and
controlling the audio signal processing unit in such a manner that, during times when no presence of an audio signal at the audio input is detected, the audio signal processing unit is operated in an ambient mode in which the audio signals captured by the left ear microphone are provided to the left ear loudspeaker and the audio signals captured by the right ear microphone are provided to the right ear loudspeaker, and during times when the presence of an audio signal at the audio input is detected, the audio signal processing unit is operated in a communication mode in which the audio signals received via the audio input are provided to at least one of the left ear loudspeaker and the right ear loudspeaker and the audio signals captured by at least one of the left ear microphone and the right ear microphone are provided to at least the other one of the left ear loudspeaker and the right ear loudspeaker.
